(19) United States
(12) Patent Application Publication
(10) Pub. No.: US 2024/0271178 A1
(43) Pub. Date: Aug. 15, 2024

(54) METHOD FOR DETECTION OF ESCHERICHIA COLI AND ANTIBIOTIC RESISTANT BACTERIA IN WATER

(71) Applicant: EASTERN KENTUCKY UNIVERSITY, Richmond, KY (US)

(72) Inventor: Jason Wayne Marion, Richmond, KY (US)

(73) Assignee: EASTERN KENTUCKY UNIVERSITY, Richmond, KY (US)

(21) Appl. No.: 17/820,452

(22) PCT Filed: Feb. 19, 2021
(86) PCT No.: PCT/US2021/018679

§ 371 Date: Aug. 17, 2022

Related U.S. Application Data
(60) Provisional application No. 62/979,694, filed on Feb. 21, 2020.

(51) Int. Cl. C12Q 1/04; G01N 33/18

(52) U.S. Cl. CPC C12Q 1/045; G01N 33/1826

(57) ABSTRACT

The present disclosure concerns systems and methods for detecting contaminants such as E. coli in water samples from rivers, lakes, or other sources of drinking water to expeditiously and inexpensively identify microbial contamination, wherein a water sample is mixed with a growth medium and a substrate to form a mixture, wherein the presence of a contaminant allows for metabolism of the substrate and detection thereof. In some aspects, equal amounts of the mixture are placed into individual wells, incubated and then visualized under UV light. Individual wells positive for the metabolite of the substrate can be counted and the number of glowing wells corresponds to the most probable number of contaminant in the sample volume.

### RELATED APPLICATIONS

This application claims priority to U.S. Provisional Patent Application 62/979,694, filed Feb. 21, 2020, the contents of which are hereby incorporated by reference in its entirety.

### FIELD OF THE DISCLOSURE

The present disclosure relates to the detection of Escherichia coli (E. coli) in water, including all sources of drinking water and recreational water. The present disclosure also relates to the detection of antibiotic resistance among E. coli bacteria in water.

### BACKGROUND

Water quality is an important public health concern worldwide, particularly in impoverished areas or areas where resources are limited. Microbial contamination of water results in significant health burdens and is often a source of disease outbreak. The World Health Organization recommends using the fecal indicator bacteria E. coli to measure the microbial quality of water. Accordingly, the level of health risks associated with any particular water source may be determined by quantifying the E. coli density in a water sample.

Unfortunately, problems of poor water quality are compounded by the lack of expedient, portable, inexpensive, and reliable methods for quantifying E. coli in water samples obtained in the field. Among other things, existing systems and methods require a means of extended refrigeration and/or power source, which may not be readily available in remote field locations. Similarly, many existing methods require expensive or specialized equipment or a laborious process that is not feasible for expansive use in the field. Likewise, other existing methods do not allow for direct quantification of E. coli but instead measure total coliform and/or require cumbersome steps that increase the likelihood of errors or cross-contamination during the testing process.

Additionally, existing methods do not specifically test for antibiotic resistance among E-coli.

Accordingly, the availability of inexpensive, portable, and expedient systems and methods for quickly detecting and quantifying E. coli and antibiotic-resistance E. coli bacteria in water from rivers, dams, ponds, lakes, and other sources of drinking water will be appreciated.

### SUMMARY OF THE DISCLOSURE

The present disclosure concerns systems and methods for determining the presence and/or quantity of contaminants in an aqueous sample. In some aspects, the present disclosure concerns a system that includes a dilution container for diluting an aliquot of the aqueous sample in a medium composition solution of a growth medium and a substrate for a contaminant. The aliquot and the medium composition solution then form a mixture and can be placed within or near an incubator for incubating the mixture for a period of time and at a temperature favorable for the contaminant to metabolize the substrate.

In some aspects, the system may also include a means dividing the mixture into 2 or more separated compartments. In certain aspects, the separated compartments may be separated wells on a plate, such as plate with 48 or 96 wells. In further aspects, the system may include a lid for the plate. In certain aspects, the system may include a means for sealing the lid.

In some aspects, the substrate of the system may be selected from 4-methylumbeliferyl-β-D-glucuronide, resorufin-β-D-glucuronide, fluorescein di-β-D-glucuronide, 5-(Pentafluorobenzoylamino)fluorescein di-β-D-glucuronide 5-bromo-6-chloro-3-indolyl-β-D-glucuronide, 5-bromo-4-chloro-3-indolyl-β-D-glucuronide, 8-hydroxyquinoline glucuronide, 4-nitrophenyl-β-D-glucuronide, phenolphthalein-β-D-glucuronide, phenolphthalein-β-D-glucuronide sodium salt, or an analog thereof.

In some aspects, the system includes a growth medium that includes sodium salts, sulfate salts, chloride salts, ammonium salts, phosphate salts, potassium salts, and/or calcium salts. In other aspects, the growth medium may include sodium sulfate, sodium chloride (or comparable sources of the ions therein), sodium phosphate, potassium phosphate, ammonium chloride, ammonium sulfate, magnesium sulfate and calcium chloride. In certain aspects, the growth medium includes from about 0.01 to about 0.1 g/mL of sodium sulfate, from about 0.01 to about 0.1 g/mL of sodium chloride, from about 0.1 to about 0.5 g/mL sodium phosphate, from about 0.1 to about 0.5 g/mL potassium phosphate, from about 0.1 to about 0.5 g/mL of ammonium chloride, from about 0.1 to about 0.5 g/mL of ammonium sulfate, from about 0.01 to about 0.05 g/mL of magnesium sulfate, and from about 0.001 to about 0.01 g/mL of calcium chloride.

In other aspects, the medium composition may further include sodium pyruvate, sodium dodecyl sulfate and/or an antibiotic. In certain aspects, the antibiotic is selected from cefsulodin, ceftazidime, cefttibuten (cedax), cefixime, cefoxitin, cefoxitin sodium salt, cefotaxime, cefotaxime sodium salt, cefoperazone, azlocillin, pipcracillin, tobramycin, and latamoxef (moxalactam), gentamicin or a combination thereof. In other aspects, the antibiotic is selected from tetracycline, glycyicycline, tigecycline, chlortetracycline, oxytetracycline, demeclocycline, lymecycline, methacycline, minocycline, rolitetracycline, doxycycline, imipenen, doripenem, meropenem, etrapenem, clisatatin or a combination thereof. In some aspects, the antibiotic is present at a concentration of from about 0.0001 g/mL to about 0.001 g/mL. In some aspects, SDS is present at a concentration of from about 0.005 to about 0.02 g/mL. In some aspects, sodium pyruvate is present at a concentration of from about 0.005 to about 0.02 g/mL.

In some aspects, the growth medium composition may include small peptides and/or amino acids and/or cellular extracts, such as yeast extract and/or casamino acids.

In some aspects, the incubator of the system provides a temperature to the mixture of from about 20 to about 40° C.

In other aspects, the present disclosure concerns methods for determining the presence and/or quantity of contaminants in an aqueous sample. The methods include forming a mixture by diluting an aliquot of an aqueous sample in a medium composition solution of a growth medium and a substrate. The methods may also include incubating the mixture for a period of time and at a temperature favorable for the contaminant to metabolize the substrate.

In some aspects, the methods may also include detecting for a metabolite of the substrate. In some aspects, the metabolite may be detected by exposing the mixture to ultraviolet light.

In some aspects, the methods may include dispensing the mixture into equal volumes in 2 or more containers before incubating the mixture, such as dispensing into 96 wells within a single plate. In certain aspects, a lid may be provided over a top surface of the plate and optionally sealed thereon.

In some aspects, the methods may include counting the wells wherein the metabolite is detected. In certain aspects, the number of wells wherein the metabolite is then extrapolated to Table 2 to determine the most probable number of contaminants per mL of the sample.

In some aspects, the methods may include incubating the mixture at a temperature of from about 20 to about 40° C. In certain aspects, the temperature is 35° C. In other aspects, the methods include incubating the mixture for a period of from about 10 to about 48 hours. In certain aspects, the period of time is about 18 hours.

In some aspects, the methods may include aspects wherein the substrate is selected from 4-methylumbeliferyl-β-D-glucuronide, resorufin-β-D-glucuronide, fluorescein di-β-D-glucuronide, 5-(Pentafluorobenzoylamino)fluorescein di-β-D-glucuronide 5-bromo-6-chloro-3-indolyl-β-D-glucuronide, 5-bromo-4-chloro-3-indolyl-β-D-glucuronide, 8-hydroxyquinoline glucuronide, 4-nitrophenyl-β-D-glucuronide, phenolphthalein-β-D-glucuronide, phenolphthalein-β-D-glucuronide sodium salt, or an analog thereof.

In some aspects, the growth medium of the methods may include sodium salts, sulfate salts, chloride salts, ammonium salts, phosphate salts, potassium salts, and/or calcium salts. In certain aspects, the growth medium includes sodium sulfate, sodium chloride (or comparable sources of the ions therein), sodium phosphate, potassium phosphate, ammonium chloride, ammonium sulfate, magnesium sulfate and calcium chloride. In further aspects, the growth medium includes from about 0.01 to about 0.1 g/mL of sodium sulfate, from about 0.01 to about 0.1 g/mL of sodium chloride, from about 0.1 to about 0.5 g/mL sodium phosphate, from about 0.1 to about 0.5 g/mL potassium phosphate, from about 0.1 to about 0.5 g/mL of ammonium chloride, from about 0.1 to about 0.5 g/mL of ammonium sulfate, from about 0.01 to about 0.05 g/mL of magnesium sulfate, and from about 0.001 to about 0.01 g/mL of calcium chloride.

In some aspects, the medium composition of the methods may include sodium pyruvate, sodium dodecyl sulfate and/or an antibiotic. In some aspects, the antibiotic is cefsulodin, ceftazidime, cefttibuten (cedax), cefixime, cefoxitin, cefoxitin sodium salt, cefotaxime, cefotaxime sodium salt, cefoperazone, azlocillin, pipcracillin, tobramycin, and latamoxef (moxalactam), gentamicin or a combination thereof. In other aspects, the antibiotic is selected from tetracycline, glycylcycline, tigecycline, chlortetracycline, oxytetracycline, demeclocycline, lynecycline, methacycline, minocycline, rolitetracycline, doxycycline, imipenem, doripenem, meropenem, erapenem, clisatatin, or a combination thereof. In certain aspects, the antibiotic is present at a concentration of from about 0.0001 g/mL to about 0.001 g/mL. In further aspects, the SDS is present at a concentration of from about 0.005 to about 0.02 g/mL. In certain aspects, sodium pyruvate is present at a concentration of from about 0.005 to about 0.02 g/mL.

In further aspects, the growth medium composition of the methods may include small peptides and/or amino acids and/or cellular extracts, such as yeast extract and/or casamino acids.

In certain aspects, the present disclosure concerns methods and systems for detecting E. coli in a water sample obtained from a household tap, rain barrel, recreational water (e.g. beach, swimming pool) cistern, groundwater source (e.g. dug well, drilled well, bore hole, etc.), surface water (e.g. river, lake, pond, wetland, dam, sea, estuary), or other source of drinking, bathing, or recreational water.

In some aspects, the systems and the methods may include one or more parameters of preparing a medium with a substrate; adding the medium to a container, such as a sterile container; adding an aliquot of a water sample to the media and mixing therein; optionally dividing the mixture into multiple fractions; incubating the mixture and determining the presence of a metabolite of the substrate therein.

In certain aspects, the medium may be prepared by (1) adding sterile, pure, clean or distilled water to a sterile container of flask. In some aspects, the container or flask may have dry goods of one or more of: yeast extract; casamino acids, sodium sulfate; sodium chloride; sodium phosphate dibasic; sodium pyruvate; potassium phosphate monobasic; ammonium chloride; ammonium sulfate; magnesium sulfate; calcium chloride; sodium dodecyl sulfate; and Cefsulodin. The medium is further prepare with a substrate such as MUG. In some aspects, the amounts of dry goods and water added are dependent upon the number of water sample tests to be performed. After combining the components of the medium, mixing may occur, such as by utilizing a magnetic stirrer on a medium to medium-high setting for one-hour.

In other aspects, after preparing the medium, a volume thereof may be transferred to a new container and mixed therein with an aliquot of a water sample to be tested. In some aspects, 2.5 mL of the liquid medium may be added to a sterile centrifuge tube utilizing sterile micropipette tips and then mixed with 22.5 mL of a water sample by capping and inverting the tube, such as about a minimum of thirty (30) times.

In further aspects, the mixture may then be dispensed into separate containers or wells. In certain aspects, such can be achieved by pouring the solution into a reservoir, such as a multichannel pipette reservoir.

In additional aspects, the mixture may be dispensed into individual wells and sealing or covering the individual wells. In certain aspects, a multichannel pipette may be utilized to efficiently withdraw a volume, such as a volume of about 200 microliters, per pipette tip from the reservoir. In further aspects, a certain volume may then be dispensed per well or container, such that each has an equal or near equal volume of the mixture. In some aspects, each well may be contained within a single plate, such as a 48 or 96 well plate. A lid may be placed thereon and optionally sealed.

In further aspects, the mixture can be incubated for a period of time at a desired temperature. In some aspects, the temperature may be of 35-44.5° C. and the period of time be of about 18 hours. In some aspects, the temperature may be of 30, 31, 32, 33, or 34° C. for a period of up to 18 hours. In other aspects, the temperature may be of 25, 26, 27, 28, or 29° C. for a period of up to about 48 hours. In further aspects, the temperature may be of 20, 21, 22, 23, 24, or 25° C. for a period of up to 60 hours.

In some aspects, after incubating the mixture a longwave light, such as ultraviolet light, may be applied to determine the presence of a metabolite of the substrate. In some aspects, the number of wells positive for the metabolite of the substrate may be counted.

In additional aspects, based on the number of wells positive for the metabolite of the substrate, the E. coli density per 100 mL of the water sample in most probable number (MPN) may be then determined.

### BRIEF DESCRIPTION OF FIGURES

FIG. 1 is a schematic view of a multichannel pipette reservoir.

FIG. 2 is a top view of a multichannel pipette withdrawing solution from a multichannel pipette reservoir.

FIG. 3 is a top view of a multichannel pipette depositing solution into a 96-well plate.

FIG. 4 is top view of an alternative means of incubation.

FIG. 5 is a top view of a 96-well plate wherein individual wells are glowing blue or turquoise.

FIG. 6 is a graphical representation of the relationship between the present disclosure and a commercially available water-testing product using the Fractional Polynomial method in Stata 15.

FIG. 7 is a schematic view of a 96-well plate and plastic adhesive seal.

FIG. 8 is a schematic view of a 96-well plate and a well lid cover.

### DETAILED DESCRIPTION

The following description illustrates embodiments sufficiently to enable those skilled in the art to practice the present disclosure. It is to be understood that the disclosure is not limited to the details of construction and the arrangement of components set forth in the following description. The disclosure is capable of being practiced or of being carried out in various ways, such as through the incorporation of structural, chronological, process, and other changes. Examples merely typify possible variations. Individual components and functions are optional unless explicitly required, and the sequence of operations may vary. Portions and features of some embodiments may be included in or substituted for those of others. The scope of the application encompasses the appended claims and all available equivalents. The following description is, therefore, not to be taken in a limited sense.

It will also be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Additionally, the words “herein,” “wherein”, “whereas”, “above,” and “below” and words of similar import, when used in this application, shall refer to this application as a whole and not to any particular portions of the application.

As used herein, the singular forms “a”, “an” and “the” include plural referents unless the context clearly dictates otherwise. “And” as used herein is interchangeably used with “or” unless expressly stated otherwise. As used herein, the term “about” means +/−5% of the recited parameter. All embodiments of any aspect of the disclosure can be used in combination, unless the context clearly dictates otherwise.

Throughout the disclosure, some percentages are presented as relative to a total weight or to a total volume.

The present disclosure concerns systems and methods for the detection and/or quantification of a microorganism/microbe/pathogen in a water sample or an aqueous sample. The systems and the methods set forth herein provide for mixing a sample with a medium composition or a medium composition solution and, following incubation, detecting the presence and/or quantity of microbes/microorganisms present in the sample. In certain aspects, the present systems provide for selection steps and/or component that allow for the detection and/or quantification of particular microorganisms or microbes in the sample. In some aspects, the present disclosure concerns methods for detecting and/or quantifying the presence of a bacterium in a sample. In some aspects, the sample is an aqueous sample, such as a water sample. In some aspects, the methods of the present disclosure allow for monitoring a water source for the presence and/or amount of bacteria therein. In certain aspects, the bacterium is Escherichia coli.

In some aspects, the present disclosure concerns medium compositions for carrying out the methods of detecting and/or quantifying a bacterium in a sample. The medium compositions, in some aspects, provide for physiological ionic conditions and/or pH conditions for bacterial growth. In other aspects, the medium compositions provide for selecting particular types and genus of bacteria.

The systems and the methods of the present disclosure require the collection of an aqueous sample to be tested from a source, such as a tap, a lake, a stream, a well and so forth. The sample to be tested can be measured to obtain an initial volume to determine or allow for a count of microbes per volume of sample. In some aspects, a desired volume of the sample can be utilized in the systems and methods herein to provide a count of microbes per assay volume of the sample and in turn per volume in the source. In such instances, it can be of benefit to agitate or mix the sample prior to providing an aliquot thereof to the systems and the methods of the present disclosure. Those skilled in the art will appreciate that any mixing or agitation should be sufficiently rigorous to allow for any settled elements to become evenly suspended without providing sufficient turbulence so as to potentially harm any microbes or microorganisms therein as such could negatively impact any accuracy provided by the systems and the methods of the present disclosure.

In certain aspects, the sample or an aliquot thereof is provided to a medium composition or a medium composition solution as described herein. In certain aspects, an aliquot or measured volume of the sample is provided to a known volume of a medium composition solution, such that a known ratio of dilution of the sample is prepared. For example, in some aspects 22.5 mL of a sample can be provided to 2.5 mL of a medium composition solution to provide a dilution ration of 10:1 of sample to medium composition solution. It will be apparent that the factor of dilution is necessary to consider when extrapolating to an estimate of a level of contamination present in the sample itself and/or in the source.

In some aspects, the system and the methods of the present disclosure concern contacting an aqueous sample with a microbial or microorganism substrate, wherein if the microorganism is present, it can metabolize the substrate. In certain aspects, metabolism of the substrate provides a detectable change in the medium composition.

In certain aspects, the present disclosure concerns a microorganism's substrate in the medium composition, wherein the presence of the microorganism leads to metabolism of the substrate. In certain aspects, the metabolized substrate is detectable, thereby allowing a user to confirm the presence of the microorganism therein. In certain aspects, the metabolized substrate is detectable by a change in color or appearance when exposed to a certain wavelength of light. In certain aspects, the metabolized substrate is detectable under visible light. In other aspects, the metabolized substrate is detectable under ultraviolet (UV) light. In further aspects, the metabolized substrate is detectable when exposed to a wavelength of light of from about 100 nm to about 400 nm, including about 150, 200, 250, 300, and 350 nm.

In certain aspects, the systems and methods of the present disclosure are designed to detect particular pathogens or microorganisms within an aqueous substrate. In certain aspects, the choice of the substrate can determine what pathogen or microorganism is being assayed for. For example, compounds such as 4-methylumbeliferyl-β-D-glucuronide (also referred to as MUG or 4-MUG) and analogs such as Resorufin-β-D-glucuronide, Fluorescein di-β-D-glucuronide, 5-bromo-6-chloro-3-indolyl-β-D-glucuronide, 5-bromo-4-chloro-3-indolyl-β-D-glucuronide, 8-hydroxyquinoline glucuronide, 4-nitrophenyl-β-D-glucuronide, phenolphthalein-β-D-glucuronide, phenolphthalein-β-D-glucuronide sodium salt, and 5-(Pentafluorobenzoylamino) fluorescein di-β-D-glucuronide, act as substrates for the 0-glucuronidase enzymes found within and/or associated with Escherichia coli. In some aspects, the substrate is provided at a concentration of from about 0.001 to about 0.01 g/mL including 0.002, 0.003, 0.004, 0.005, 0.006, 0.007, 0.008, and 0.009 g/mL.

In further aspects, other selection components may be utilized with the systems and methods of the present disclosure. For example, while E. coli may be the primary microorganism that metabolizes substrates such as MUG, other microorganisms such as Pseudomonas may also metabolize the substrate. Accordingly, other selection components may be included to increase the specificity. For example, inclusion of sodium dodecyl sulfate (SDS) can inhibit activity and/or presence of gram-positive bacteria. Inclusion of antibiotics such as cephalosporins can selectively act on microbes such as Pseudomonas within the sample to remove them from exerting any effect on the substrate. In some aspects, the cephalosporin is a third generation cephalosporin. In further aspects, the antibiotic is cefsulodin, ceftazidime, cefttibuten (cedax), cefixime, cefoxitin, cefoxitin sodium salt, cefotaxime, cefotaxime sodium salt, cefoperazone, azlocillin, pipcracillin, tobramycin, and latamoxef (moxalactam). In further aspects, gentamicin may be added to the antibiotic. In certain aspects, the antibiotic is cefsulodin. In further aspects, cefsulodin can be utilized in at a concentration of from about 0.0001 g/mL to about 0.001 g/mL including 0.0002, 0.0003, 0.0004, 0.0005, 0.0006, 0.0007, 0.0008, and 0.0009 g/mL.

Similarly, other antibiotics can be utilized within the system and the methods of the present disclosure. It is understood that antibiotics have a spectrum of activity and utilizing the spectra available can allow for selection of particular microorganisms. For example, cefsulodin is understood to have activity to Pseudomonas but not E. coli. Conversely, ceftriaxone has poor activity against Pseudomonas but can exert activity against E. coli and other gram-negative bacilli. Penicillin can select to exclude Streptococci and clindamycin can select to exclude gram-positive cocci in general. In certain aspects, such further selection components can be used in subsequent assays to better determine the microorganisms present in the sample and/or the source of the sample.

In is also understood that some microorganisms can exhibit resistance to some antibiotics. Obtaining such information can be of benefit to the population surrounding the source of the sample such that the community in general can gain an understanding of what agents to utilize to better treat anyone suspected of succumbing to an infection. For example, E. coli can frequently become resistant to agents such as tetracycline. Accordingly, either in an initial run or in a subsequent run, tetracycline can be utilized within the systems and the methods of the present disclosure to allow for a determination of the presence of a resistant microorganism and/or the quantification of such a resistant microorganism within the sample. In some aspects, tetracycline can be utilized at from about 0.0001 to about 0.001 g/mL, including about 0.0002, 0.0003, 0.0004, 0.0005, 0.0006, 0.0007, 0.0008, and 0.0009 g/mL. In other aspects, other agents for resistance can be utilized, including a glycylcycline subclass agent (tigecycline), chlortetracycline, oxytetracycline, deneclocycline, and semi-synthetic tetracyclines including lymecycline, methacycline, minocycline, rolitetracycline, and doxycycline. In further aspects, the system can include an agent to select for multi-drug resistance with the inclusion of a carbapenem, such as imipenem, doripenem, meropenem, etrapenem, and clisatatin. In some aspects, clisatatin can be used in combination with imipenem. In other aspects, the system may incorporate agents to evaluate for the presence of extended-spectrum enzymes, such as beta-lactamases. In such aspects, the systems may further include cefotaxime or cefotaxime and clavulanic acid or ceftazidime or ceftazidime and clavulanic acid.

As set forth herein, an aliquot of the sample is diluted within a medium composition. In aspects, the medium composition includes a growth medium for any potential contaminants. The medium composition can be a pre-made aqueous solution or a massed preparation of dry goods collected in one or more sets or each component isolated until the medium composition is required for use and therein dissolved in an appropriate volume of water. As the system and methods described herein concern determining the presence and/or quantity of a contaminant microorganism, use of sterile or purified water can be beneficial for accuracy. In some aspects, it will be apparent that certain components of the medium composition and/or system and methods as set forth herein can deteriorate and/or degrade over time when in solution or exposed to light or heat and similar. In those aspects at least, it can be of benefit to add those components as close to the point of use as possible. For example, the medium composition can include sodium and chloride, which can be a singular component as sodium chloride, which is understood to have good tolerance in solution. As such, a need to add sodium chloride closer to a time of use of less importance. However, components such as antibiotics and/or the substrate component can potentially deteriorate or degrade in solution and can be of more effective use if provided to the system and methods at a point in time close to use thereof.

It will also be appreciated that providing a growth medium within a medium composition as an aqueous solution provides an attractive medium for ambient microorganisms. Accordingly, it will be understood that precautions such as sterile technique and maintaining aseptic conditions will be of benefit to avoid contamination and ensuing inaccuracies. In certain environments, it may further be necessary to reconstitute the medium composition in an aqueous solution to a point in time close to use in order to avoid contamination.

In certain aspects of the present disclosure, the sample is mixed and diluted within a medium composition solution. In some aspects, the medium composition is provided as either a dry good or as an aqueous solution. For the purposes of dilution, the medium composition as a dry good should be reconstituted in an aqueous solution prior to use within the systems and methods set forth herein. Further, as the medium compositions are to determine the presence or absence of microbes and/or pathogens in an aqueous sample, care should be taken into preparing the medium composition solutions, either at the point of testing or anytime there before to avoid contamination.

In some aspects, the growth medium within the medium composition can be a mixture of dried salts of anions and cations that can be dissolved into an aqueous medium composition solution for assaying a water sample or an aqueous sample for the presence of a microbe or microorganism or pathogen therein. Such may include sodium salts, sulfate salts, chloride salts, ammonium salts, phosphate salts, pyruvate salts, potassium salts, and/or calcium salts. Such may also include a surfactant, such as an anionic surfactant, including sodium dodecyl sulfate. Such may also include small peptides and/or amino acids and/or cellular extracts, such as yeast extract. Such may also include antibiotics. In some aspects, “dry goods” may also include one or more of the following: yeast extract, cas-amino acids, sodium sulfate (or comparable sources of the ions therein), sodium chloride (or comparable sources of the ions therein), sodium phosphate dibasic (or comparable sources of the ions therein), sodium pyruvate (or comparable sources of the ions therein), potassium phosphate monobasic (or comparable sources of the ions therein), ammonium chloride (or comparable sources of the ions therein), ammonium sulfate (or comparable sources of the ions therein), magnesium sulfate (or comparable sources of the ions therein), calcium chloride (or comparable sources of the ions therein).

In further aspects, the medium composition can including selection agents as described herein including sodium dodecyl sulfate (or similar anionic surfactant) to inhibit gram-positive bacteria growth and/or activity and/or cefsulodin to inhibit Pseudomonas contamination and/or tetracycline to identify resistant strains. In further aspects, the medium composition includes the substrate for the microorganism, such as MUG or an equivalent thereof.

In some aspects, the growth medium of the medium compositions include sodium and/or potassium and/or ammonium and/or magnesium and/or calcium cations. In other aspects, the growth medium of the medium compositions include sulfate and/or chloride and/or pyruvate and/or phosphate anions. In some aspects, the growth medium of the medium compositions contain from about 0.05 to about 0.25 moles of sodium cations, including about 0.06, 0.07, 0.08, 0.09, 0.1, 0.11, 0.12, 0.13, 0.14, 0.15, 0.16, 0.17, 0.18, 0.119, 0.2, 0.21, 0.22, 0.23, and 0.24 moles of sodium. In some aspects, the growth medium of the medium compositions contain from about 0.01 to about 0.1 moles of potassium cations, including about 0.02, 0.03, 0.04, 0.05, 0.06, 0.07, 0.08, and 0.09 moles. In some aspects, the growth medium of the medium compositions include from about 0.05 to about 0.25 moles of ammonium cations, including about 0.06, 0.07, 0.08, 0.09, 0.1, 0.11, 0.12, 0.13, 0.14, 0.15, 0.16, 0.17, 0.18, 0.119, 0.2, 0.21, 0.22, 0.23, and 0.24 moles of ammonium. In some aspects, the growth medium of the medium compositions include from about 0.001 to about 0.02 moles of magnesium cations, including about 0.002, 0.003, 0.004, 0.005, 0.006, 0.007, 0.008, 0.009, 0.01, 0.011, 0.012, 0.013, 0.014, 0.015, 0.016, 0.017, 0.018, and 0.019 moles of magnesium. In other aspects, the growth medium of the medium compositions may include from about 0.0005 to about 0.002 moles of calcium cations, including about 0.0006, 0.0007, 0.0008, 0.0009, 0.001, 0.0011, 0.0012, 0.0013, 0.0014, 0.0015, 0.0016, 0.0017, 0.0018, and 0.0019 moles of calcium.

In some aspects the growth medium of the medium compositions may include from about 0.005 to about 0.05 moles of sulfate anions, including 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, and 0.049 moles of sulfate. In some aspects, the growth medium of the medium compositions include from about 0.001 to about 0.7 moles of chloride anions, including about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, and 0.69 moles of chloride. In some aspects, the growth medium of the medium compositions may include from about 0.01 to about 0.1 moles of phosphate anions, including 0.02, 0.03, 0.04, 0.05, 0.06, 0.07, 0.08, and 0.09 moles of phosphate. In some aspects, the growth medium of the medium compositions may include from about 0.001 to about 0.01 moles of pyruvate anions, including about 0.002, 0.003, 0.004, 0.005, 0.006, 0.007, 0.008, and 0.009 moles of pyruvate.

In further aspects, the growth medium of the medium composition may provide a certain molarity of the cations and anions as set forth herein. It will be apparent to those skilled in the art that based on a particular molarity or range thereof that the quantity of each cation and anion can be adjusted such that the required amount for a desired volume is provided. In some aspects the growth medium of the medium composition has a sodium molarity of from about 0.2 M to about 1.15 M, a potassium molarity from about 0.04 M to about 0.45 M, an ammonium molarity of from about 0.2 M to about 1.15 M, a magnesium molarity of from about 0.004 M to about 0.1 M, a calcium molarity of from about 0.002 M to about 0.01 M, a sulfate molarity of from about 0.02 M to about 0.25 M a chloride molarity of from about 0.004 M to about 3.45 M a phosphate molarity of from about 0.04 M to about 0.45 M and a pyruvate molarity of from about 0.04 M to about 0.045 M.

In further aspects, the growth medium of the medium composition may include a mixture of salts of varying anions and cations as set forth herein. In some aspects, the growth medium of the medium composition may be of about 0.01 to about 0.1 g/mL of sodium sulfate (including about 0.02, 0.03, 0.04, 0.05, 0.06, 0.07, 0.08 and 0.09) and/or about 0.01 to about 0.1 g/mL of sodium chloride (including about 0.02, 0.03, 0.04, 0.05, 0.06, 0.07, 0.08 and 0.09) and/or about 0.1 to about 0.5 g/mL sodium phosphate (monobasic or dibasic) (including about 0.2, 0.3, and 0.4) and/or about 0.1 to about 0.5 g/mL potassium phosphate (monobasic) (including about 0.2, 0.3, and 0.4) and/or about 0.1 to about 0.5 g/mL of ammonium chloride (including about 0.2, 0.3, and 0.4) and/or about 0.1 to about 0.5 g/mL of ammonium sulfate (including about 0.2, 0.3, and 0.4) and/or about 0.01 to about 0.05 g/mL of magnesium sulfate (including about 0.02, 0.03, and 0.04) and/or about 0.001 to about 0.01 g/mL of calcium chloride (including about 0.002, 0.003, 0.004, 0.005, 0.006, 0.007, 0.008 and 0.009). In further aspects, the medium composition may further include SDS at about 0.005 to about 0.02 g/mL (including about 0.006, 0.007, 0.008, 0.009, 0.01, 0.011, 0.012, 0.013, 0.014, 0.015, 0.016, 0.017, 0.018, and 0.019) and/or sodium pyruvate at about 0.005 to about 0.02 g/mL (including about 0.006, 0.007, 0.008, 0.009, 0.01, 0.011, 0.012, 0.013, 0.014, 0.015, 0.016, 0.017, 0.018, and 0.019).

In some aspects, the growth medium of the medium compositions may include amino acids and/or cellular extracts. For example, the presence of amino acids can provide basic nutrients to any present microbes to prevent such from escaping detection in the system and the methods herein. The presence of amino acids and/or cellular extracts could potentially provide for an environment that can resemble that in which a microbe might seek or be capable of survival in, such that the microbe avoids cell death prior to completion of the steps to quantify such as set forth herein. In some aspects, the medium compositions can include from about 0.1 to about 0.7 g/mL of a yeast extract, including about 0.2, 0.3, 0.4, 0.5, and about 0.6. The medium compositions may include from about 0.05 to about 0.5 g/mL of an amino acid composition, including about 0.1, 0.15, 0.2, 0.25, 0.3, 0.35, 0.4, and 0.45 g/mL. In certain aspects the amino acids compositions may include amino acids and/or peptides derived from hydrolysis of a common protein such as casein (referred herein as “casamino acids”).

In further aspects, the growth medium of the medium composition can include basic salts and amino acids to allow for the microorganism(s) to grow therein during use of the systems and the methods set forth herein. In some aspects, the medium composition can be based on a minimal salts medium (Roberts et al., Studies of the biosynthesis of Escherichia coli, 1955) and/or similar growth media (Bain et al. PLoS ONE 10(10): e0140997, 2015).

In certain aspects, the medium composition can include the following for the indicated final mL of medium composition solution as set forth in Table 1:

TABLE 1

| | g/22.5 mL | g/100 mL | g/mL |
|---|---|---|---|
| Yeast Extract | 5 | 22.22225 | 0.2222225 |
| Casamino Acids | 2.5 | 11.111125 | 0.11111125 |
| Sodium Sulfate | 1.25 | 5.5555625 | 0.055555625 |
| Sodium Chloride | 1.25 | 5.5555625 | 0.055555625 |
| Sodium Phosphate Dibasic | 7.5 | 33.333375 | 0.33333375 |
| Sodium Pyruvate | 0.25 | 1.1111125 | 0.011111125 |
| Potassium Phosphate | 3.75 | 16.6666875 | 0.166666875 |
| Monobasic | | | |
| Ammonium Chloride | 2.5 | 11.111125 | 0.11111125 |
| Ammonium Sulfate | 2.5 | 11.111125 | 0.11111125 |
| Magnesium Sulfate | 0.625 | 2.77778125 | 0.027777813 |
| Calcium Chloride | 0.125 | 0.55555625 | 0.005555563 |
| Sodium Dodecyl Sulfate | 0.25 | 1.1111125 | 0.011111125 |
| MUG | 0.125 | 0.55555625 | 0.005555563 |
| Cefsulodin | 0.015 | 0.06666675 | 0.000666668 |
| *Tetracycline | 0.016 | 0.0711112 | 0.000711112 |
| Optional Sterile | 2.5 | 11.111125 | 0.11111125 |
| Water or Sterile | | | |
| Buffer Solution | | | |
| or Sterile Saline | | | |
| (2.5 mL) | | | |
| Sample Volume (mL = g) | 22.5 | 100.000125 | 1.00000125 |

In some aspects, the present disclosure concerns systems and methods that allow for determining the presence of microbes or microorganisms within a sample. In other aspects, the compositions, as well as the methods of use thereof, allow for an accurate determination of the number of bacteria or a most probable number thereof within a sample. The process for determining the presence and/or quantity can depend on a number of contributing factors depending on the materials available to a user. Accordingly, while the examples herein demonstrate success with certain sets of dilutions, incubation times, incubation temperatures and further variable parameters, it will be apparent to those skilled in the art that the steps can be readily modified and/or adjusted to accommodate other desired processes and/or conditions.

As set forth in the working examples, an aqueous sample is obtained from a source and an aliquot thereof is mixed with the medium composition. As identified herein, the medium composition may include certain levels of anions, cations, amino acids/peptides, and selection components. As illustrated in the examples, the aliquot is diluted as one part medium in 10 parts sample. It will be apparent that other levels of dilution can be utilized, such as 1:1, 1:2, 1:3, 1:4, 1:5, 1:6, 1:7, 1:8, 1:9, 1:20, 1:30, 1:40, 1:50, 1:60, 1:70, 1:80, 1:90, 1:100, 1:1000, 1:10000, 1:100000 and so on, including intermediates there between. In some aspects, it a sample can be diluted at varying ratios and proceed with each. For example, the methods can be carried out with a 1:2 dilution and 1:10 dilution and so forth in parallel assays. Such can provide a further control, as well as account for a particularly high or low number of microorganisms present in the source.

In some aspects, the mixed composition of the sample aliquot diluted in the medium composition solution can then been either incubated alone in a container or divided into equal or nearly equal parts. In some aspects, leaving the mixed composition as a single diluted mixture can provide for a determination of the presence of a microorganism therein. Further, if certain selection components are utilized, it can be determined, for example, that the contaminant is not Pseudomonas and/or that the contaminant is tetracycline resistant. In further aspects, dividing the mixed composition into equal or near-equal parts allows for a quantification aspect of the contaminant.

As set forth in the working examples, the mixed composition can be evenly divided into multiple parts. As an example of convenience, a 96-well plate is utilized in the working examples with a multi-channel pipette utilized for even and rapid dispensing. However, it will be apparent that other receptacles and means for parsing the mixed composition can be utilized, including smaller well numbers or individual containers or receptacles. As set forth in the examples herein, the division into multiple parts can result in a mixture of responses, i.e. some parts have sufficient contaminants present therein to grow and metabolize the substrate such that it can be detected as its metabolite, while others contain insufficient contaminants such that the growth and activity is not sufficient to detect the conversion to the metabolite of the substrate. As set forth herein, based on the number of divided parts, a clearer calculation or estimation of the amount of contaminants therein is achieved.

In some aspects, the parameters of the methods and operating conditions of the systems herein can be adjusted or altered. The systems and the methods presented herein require time for both contaminant growth and contaminant activity to allow for the metabolism of the substrate. As set forth in the working examples, E coli can be assayed and calculated by allowing for growth at temperatures of around 35° C. for 18 hours or similar, such as under exposure to a 100 W light bulb. Such accordingly provides for particular conditions to allow for a reliable growth time or rate of doubling. It will be apparent to those in the art that lower temperatures can reduce the growth rate, while higher temperatures may increase doubling or, if sufficiently increased, harm or kill the contaminant. As such, while the working examples herein are presented under particular conditions that can be easily replicated, it is to also be understood that variance of the incubation conditions is also herein contemplated. For example, the mixed compositions can be incubated for periods of about 10 hours up to about 48 hours, including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and 47 hours. The mixed compositions can be incubated at a temperature of from about 15° C. to about 40° C., including about 16, 17, 18, 19, 20, 21, 22, 23, 24, 25, 26, 27, 28, 29, 30, 31, 32, 33, 34, 35, 36, 37, 38, and 39° C. It will be appreciated that such variance can affect the growth rate and as such would require appropriate adjustment to the steps set forth herein to determine the concentration of contaminants in the sample.

It will also be apparent that providing a closure or lid to each well or receptacle can prevent contamination from the ambient atmosphere. As such, while not required, such can be of use for higher accuracy. Further, once provided to each well or receptacle, it is a further option to seal either each well or container or each tray or array of wells for further accuracy purposes.

As set forth herein, the mixed composition is divided in parts to determine not only the presence of contaminants but also the quantity present in the sample. Through the steps of dilution within the medium composition and the division into equal or near-equal parts, the contaminants are diluted such that each part may have unequal concentrations of contaminant therein. In aspects where the source is highly contaminated or suspected of being highly contaminated, a step of further dilution as set forth herein may be advisable. For example, if the sample is highly contaminated with E. coli, a low level of dilution will still provide a high number of bacteria to each divided part and with a high starting number in each part, it can be expected that each part will experience exponential growth during the incubation period and provide nothing but positive results for each part.

The present disclosure provides in part for an estimation of contaminant concentration within a sample. The working examples herein provide for a system and method for estimating the presence of E. coli in a water sample. The working examples further provide both incubation and dilution conditions that can be readily replicated with minimal sterile technique and provide counts within the realm of concern for public health for E coli contaminations. For example, the working examples provide for systems and methods that require inexpensive materials, relying on a multi-welled plate and an incubation time of about 18 hours at 37° C., thereby allowing for the system to be provided to any user and accurately executed with a high certainty of results. As the working examples include steps that take into account the concentration levels of public health concern for E. coli contamination. As explained herein, the dilutions and incubation conditions can be adjusted as needed.

In aspects, the present disclosure concerns a system for determining the presence and/or quantity of a microorganism contamination at a source. In some aspects, the contaminant is E. coli. The system provides for providing an aliquot of a sample from the source and diluting the aliquot with a medium composition solution that provides ions and amino acids to allow for the potential contaminant to grow. Also introduced within the mixture of the aliquot and the medium composition is a substrate for an enzyme particular to the suspected contaminant. In some aspects, if the suspected contaminant is E. coli, the substrate is MUG or an analog thereof. In some aspects, the mixture of aliquot and the medium composition is then incubated at a temperature and for a designated period of time that will allow for sufficient growth of a threshold count of bacteria such that if a higher suspected number is present, the substrate will be sufficiently metabolized during the incubation to provide a detectable change. In some aspects, such as when the substrate is MUG, the detectable change is a visual change under UV light.

In other aspects, the aliquot is mixed with the medium composition and substrate and divided into equal or near-equal parts. In some aspects, the mixture can be divided into multiple wells or containers. In the aspects herein concerning the accounting of E. coli, a 96 well plate is utilized following a 10:1 dilution of the sample aliquot in the medium composition solution. The division and dilution, as well as the described 18 hour incubation at 35° C. provide conditions to calculate the amount or the most probable number (MPN) of E. coli present in the obtained sample. In using these parameters, it is then possible to determine the quantity or MPN based on the number of wells positive for the metabolized substrate. As is understood, the substrate of MUG or analogs thereof results is a detectable color change, particularly when exposed to UV light. Based on these particular conditions, a MPN value based on the number of glowing wells is set forth in Table 2:

TABLE 2

| Number of | |
|---|---|
| Wells | MPN |
| Glowing | Range |
| 0 | 0-14 |
| 1 | 12-15 |
| 2 | 31-34 |
| 3 | 41-46 |
| 4 | 49-54 |
| 5 | 54-60 |
| 6 | 60-65 |
| 7 | 62-69 |
| 8 | 65-73 |
| 9 | 68-76 |
| 10 | 71-79 |
| 11 | 73-81 |
| 12 | 75-84 |
| 13 | 77-86 |
| 14 | 80-89 |
| 15 | 82-91 |
| 16 | 84-94 |
| 17 | 86-96 |
| 18 | 89-99 |
| 19 | 91-101 |
| 20 | 94-104 |
| 21 | 97-107 |
| 22 | 99-110 |
| 23 | 103-113 |
| 24 | 106-117 |
| 25 | 109-121 |
| 26 | 113-125 |
| 27 | 117-130 |
| 28 | 121-134 |
| 29 | 125-139 |
| 30 | 130-144 |
| 31 | 135-149 |
| 32 | 139-155 |
| 33 | 145-161 |
| 34 | 150-167 |
| 35 | 157-173 |
| 36 | 162-180 |
| 37 | 169-187 |
| 38 | 176-195 |
| 39 | 183-203 |
| 40 | 191-211 |
| 41 | 199-220 |
| 42 | 207-229 |
| 43 | 215-239 |
| 44 | 224-249 |
| 45 | 234-259 |
| 46 | 244-270 |
| 47 | 254-281 |
| 48 | 264-293 |
| 49 | 276-305 |
| 50 | 287-318 |
| 51 | 299-331 |
| 52 | 311-345 |
| 53 | 324-359 |
| 54 | 338-373 |
| 55 | 351-374 |
| 56 | 366-405 |
| 57 | 381-421 |
| 58 | 396-438 |
| 59 | 412-456 |
| 60 | 428-474 |
| 61 | 445-493 |
| 62 | 463-512 |
| 63 | 481-532 |
| 64 | 499-552 |
| 65 | 518-573 |
| 66 | 538-595 |
| 67 | 558-618 |
| 68 | 579-641 |
| 69 | 601-665 |
| 70 | 623-689 |
| 71 | 646-714 |
| 72 | 669-740 |
| 73 | 693-767 |
| 74 | 718-794 |
| 75 | 743-822 |
| 76 | 770-851 |
| 77 | 796-881 |
| 78 | 824-911 |
| 79 | 853-942 |
| 80 | 881-974 |
| 81 | 910-1007 |
| 82 | 940-1040 |
| 83 | 972-1074 |
| 84 | 1003-1109 |
| 85 | 1036-1145 |
| 86 | 1069-1182 |
| 87 | 1103-1220 |
| 88 | 1138-1258 |
| 89 | 1174-1298 |
| 90 | 1210-1338 |
| 91 | 1247-1379 |
| 92 | 1285-1421 |
| 93 | 1324-1464 |
| 94 | 1364-1508 |
| 95 | 1404-1553 |
| 96 | >1404 |

The numbers provided in Table 1 were generated using a ±5% to a fitted model. The fitted model was calculated by obtaining the number of glowing wells, then adding one and dividing by ten to provide term x. A first term was then generated by the equation of: x^{(-0.5})−0.485111432, and a second term was generated of x³−76.7274711. The MPN fitted model was calculated as a sum of (term 1*−45.92822) and (term 2*1.556309) and 213.7125. The models for the shaped used in the fitted model are set forth in Table 3:

TABLE 3
. fracpoly regress bestfits glow_wells95, compare
−> gen double Iglow_{——}1 = X\hat{ }−.5 − .4851114432 if e (sample)
−> gen double Iglow_{——}2 = X\hat{ }3 − 76.72747111 if e (sample)
(where: X = (glow_wells95 + 1)/10)

| Source | SS | df | MS | |
|---|---|---|---|---|
| Model | 13785424.6 | 2 | 6892712.32 | Number of obs = 71 |
| Residual | 3451.9133 | 68 | 50.7634308 | F(2, 68) > 99999.00 |
| Total | 13788876.6 | 70 | 196983.951 | Prob > F = 0.0000 |

| | | | | | R-squared = 0.9997 | |
|---|---|---|---|---|---|---|
| | | | | | Adj R-squared = 0.9997 | |
| | | | | | Root MSE = 7.1248 | |
| bestfits | Coef. | Std. Err. | t | P > \|t\| | [95% Conf. Intercal] | |
| Iglow_{——}1 | −45.92822 | 2.04379 | −22.47 | 0.000 | −50.00653 | −41.8499 |
| Iglow_{——}2 | 1.556309 | .0035254 | 441.46 | 0.000 | 1.549274 | 1.563344 |
| _cons | 213.7125 | 1.118028 | 191.15 | 0.000 | 211.4815 | 215.9435 |

Deviance: 477.25. Best powers of glow_wells95 among 44 models fit: −.53.

Fractional polynomial model comparisons :

| glow wells95 | df | Deviance | Res. SD | Dev. dif. | P (*) | Powers |
|---|---|---|---|---|---|---|
| Not in model | 0 | 1066.034 | 443.829 | 588.781 | 0.000 | |
| Linear | 1 | 918.054 | 157.672 | 440.801 | 0.000 | 1 |
| m = 1 | 2 | 628.581 | 20.5318 | 151.327 | 0.000 | 3 |
| m = 2 | 4 | 477.254 | 7.12485 | — | — | −.53 |

(*) P-value from deviance difference comparing reported model with m = 2 model

### Aspects

In a 1^{st} aspect, either alone or in combination with any other aspect herein, the present disclosure is directed to a system for determining the presence and/or quantity of contaminants in an aqueous sample comprising a dilution container for diluting a aliquot of the aqueous sample in a medium composition solution comprising a growth medium and a substrate for a contaminant to form a mixture and an incubator for incubating the mixture for a period of time and at a temperature favorable for the contaminant to metabolize the substrate.

In a 2^{nd} aspect, either alone or in combination with any other aspect herein, the present disclosure is directed to the system of the 1^{st} aspect, further comprising a means dividing the mixture into 2 or more separated compartments.

In a 3^{rd} aspect, either alone or in combination with any other aspect herein, the present disclosure is directed to the system of the 2^{nd} aspect, wherein the separated compartments are separated wells on a plate.

In a 4^{th} aspect, either alone or in combination with any other aspect herein, the present disclosure is directed to the system of the 3^{rd} aspect, wherein the plate comprises forty eight wells.

In a 5^{th} aspect, either alone or in combination with any other aspect herein, the present disclosure is directed to the system of the 3^{rd} or 4^{th} aspect, wherein the plate comprises ninety-six wells.

In a 6^{th} aspect, either alone or in combination with any other aspect herein, the present disclosure is directed to the system of any of aspects 3 to 5, further comprising a lid for the plate.

In a 7^{th} aspect, either alone or in combination with any other aspect herein, the present disclosure is directed to the system the 6^{th} aspect, wherein the lid is sealed to the plate.

In an 8^{th} aspect, either alone or in combination with any other aspect herein, the present disclosure is directed to the system of the 1^{st} aspect, wherein the substrate is selected from 4-methylumbeliferyl-β-D-glucuronide, resorufin-β-D-glucuronide, fluorescein di-β-D-glucuronide, 5-bromo-6-chloro-3-indolyl-β-D-glucuronide, 5-bromo-4-chloro-3-indolyl-β-D-glucuronide, 8-hydroxyquinoline glucuronide, 4-nitrophenyl-β-D-glucuronide, phenolphthalein-β-D-glucuronide, phenolphthalein-β-D-glucuronide sodium salt, and 5-(Pentafluorobenzoylamino)fluorescein di-β-D-glucuronide or an analog thereof.

In a 9^{th} aspect, either alone or in combination with any other aspect herein, the present disclosure is directed to the system of the 1^{st} aspect, wherein the growth medium comprises sodium salts, sulfate salts, chloride salts, ammonium salts, phosphate salts, potassium salts, and/or calcium salts.

In a 10^{th} aspect, either alone or in combination with any other aspect herein, the present disclosure is directed to the system of the 9^{th} aspect, wherein the growth medium comprises sodium sulfate, sodium chloride (or comparable sources of the ions therein), sodium phosphate, potassium phosphate, ammonium chloride, ammonium sulfate, magnesium sulfate and calcium chloride.

In an 11^{th} aspect, either alone or in combination with any other aspect herein, the present disclosure is directed to the system of the 10^{th} aspect, wherein the growth medium comprises from about 0.01 to about 0.1 g/mL of sodium sulfate, from about 0.01 to about 0.1 g/mL of sodium chloride, from about 0.1 to about 0.5 g/mL sodium phosphate, from about 0.1 to about 0.5 g/mL potassium phosphate, from about 0.1 to about 0.5 g/mL of ammonium chloride, from about 0.1 to about 0.5 g/mL of ammonium sulfate, from about 0.01 to about 0.05 g/mL of magnesium sulfate, and from about 0.001 to about 0.01 g/mL of calcium chloride.

In a 12^{th} aspect, either alone or in combination with any other aspect herein, the present disclosure is directed to the system of any previous aspect, wherein the medium composition further comprising sodium pyruvate, sodium dodecyl sulfate and/or an antibiotic.

In a 13^{th} aspect, either alone or in combination with any other aspect herein, the present disclosure is directed to the system of the 12^{th} aspect, wherein the antibiotic is cefsulodin.

In a 14^{th} aspect, either alone or in combination with any other aspect herein, the present disclosure is directed to the system of the 12^{th} aspect, wherein the antibiotic is tetracycline.

In a 15^{th} aspect, either alone or in combination with any other aspect herein, the present disclosure is directed to the system of aspects 12, 13, or 14, wherein the antibiotic is present at a concentration of from about 0.0001 g/mL to about 0.001 g/mL.

In a 16^{th} aspect, either alone or in combination with any other aspect herein, the present disclosure is directed to the system of the 12^{th} aspect, wherein SDS is present at a concentration of from about 0.005 to about 0.02 g/mL.

In a 17^{th} aspect, either alone or in combination with any other aspect herein, the present disclosure is directed to the system of the 12^{th} aspect, wherein sodium pyruvate is present at a concentration of from about 0.005 to about 0.02 g/mL.

In an 18^{th} aspect, either alone or in combination with any other aspect herein, the present disclosure is directed to the system any previous aspect, wherein the growth medium composition comprises small peptides and/or amino acids and/or cellular extracts.

In a 19^{th} aspect, either alone or in combination with any other aspect herein, the present disclosure is directed to the system of the 18^{th} aspect, wherein the growth medium composition comprises yeast extract and/or casamino acids.

In a 21^{st} aspect, either alone or in combination with any other aspect herein, the present disclosure is directed to the system any previous aspect, wherein the incubator provides a temperature to the mixture of from about 20 to about 40° C.

In a 22^{nd} aspect, either alone or in combination with any other aspect herein, the present disclosure is directed to a method for determining the presence and/or quantity of contaminants in an aqueous sample comprising forming a mixture by diluting a aliquot of an aqueous sample in a medium composition solution comprising a growth medium and a substrate and incubating the mixture for a period of time and at a temperature favorable for the contaminant to metabolize the substrate.

In a 23^{rd} aspect, either alone or in combination with any other aspect herein, the present disclosure is directed to the method of the 22^{nd} aspect, further comprising detecting for a metabolite of the substrate.

In a 24^{th} aspect, either alone or in combination with any other aspect herein, the present disclosure is directed to the method of 23^{rd} aspect, wherein the metabolite is detected by exposing the mixture to ultraviolet light.

In a 25^{th} aspect, either alone or in combination with any other aspect herein, the present disclosure is directed to the method of aspects 22 to 24, wherein the mixture is dispensed into equal volumes in 2 or more containers before incubating the mixture.

In a 26^{th} aspect, either alone or in combination with any other aspect herein, the present disclosure is directed to the method of the 25^{th} aspect, wherein the mixture is dispensed into 96 wells within a single plate.

In a 27^{th} aspect, either alone or in combination with any other aspect herein, the present disclosure is directed to the method of the 26^{th} aspect, wherein a lid is provided over a top surface of the plate.

In a 28^{th} aspect, either alone or in combination with any other aspect herein, the present disclosure is directed to the method the 26^{th} aspect, further comprising counting the wells wherein the metabolite is detected.

In a 29^{th} aspect, either alone or in combination with any other aspect herein, the present disclosure is directed to the method of the 28^{th} aspect, wherein the number of wells wherein the metabolite is extrapolated to Table 2 to determine the most probable number of contaminants per mL of the sample.

In a 30^{th} aspect, either alone or in combination with any other aspect herein, the present disclosure is directed to the method of aspects 22-29, wherein the mixture is incubated at a temperature of from about 20 to about 40° C.

In a 31^{st} aspect, either alone or in combination with any other aspect herein, the present disclosure is directed to the method of the 30^{th} aspect, wherein the temperature is 35° C.

In a 32^{nd} aspect, either alone or in combination with any other aspect herein, the present disclosure is directed to the method of any of aspects 22-31, wherein the mixture is incubated for a period of from about 10 to about 48 hours.

In a 33^{rd} aspect, either alone or in combination with any other aspect herein, the present disclosure is directed to the method of the 32^{nd} aspect, wherein the period of time is about 18 hours.

In a 34^{th} aspect, either alone or in combination with any other aspect herein, the present disclosure is directed to the method of any of aspects 22-33, wherein the substrate is selected from 4-methylumbeliferyl-β-D-glucuronide, resorufin-β-D-glucuronide, fluorescein di-β-D-glucuronide, 5-bromo-6-chloro-3-indolyl-β-D-glucuronide, 5-bromo-4-chloro-3-indolyl-β-D-glucuronide, 8-hydroxyquinoline glucuronide, 4-nitrophenyl-β-D-glucuronide, phenolphthalein-β-D-glucuronide, phenolphthalein-β-D-glucuronide sodium salt, and 5-(Pentafluorobenzoylamino)fluorescein di-β-D-glucuronide or an analog thereof.

In a 35^{th} aspect, either alone or in combination with any other aspect herein, the present disclosure is directed to the method of any of aspects 22-34, wherein the growth medium comprises sodium salts, sulfate salts, chloride salts, ammonium salts, phosphate salts, potassium salts, and/or calcium salts.

In a 36^{th} aspect, either alone or in combination with any other aspect herein, the present disclosure is directed to the method of the 35^{th} aspect, wherein the growth medium comprises sodium sulfate, sodium chloride (or comparable sources of the ions therein), sodium phosphate, potassium phosphate, ammonium chloride, ammonium sulfate, magnesium sulfate and calcium chloride.

In a 37^{th} aspect, either alone or in combination with any other aspect herein, the present disclosure is directed to the method of the 36^{th} aspect, wherein the growth medium comprises from about 0.01 to about 0.1 g/mL of sodium sulfate, from about 0.01 to about 0.1 g/mL of sodium chloride, from about 0.1 to about 0.5 g/mL sodium phosphate, from about 0.1 to about 0.5 g/mL potassium phosphate, from about 0.1 to about 0.5 g/mL of ammonium chloride, from about 0.1 to about 0.5 g/mL of ammonium sulfate, from about 0.01 to about 0.05 g/mL of magnesium sulfate, and from about 0.001 to about 0.01 g/mL of calcium chloride.

In a 38^{th} aspect, either alone or in combination with any other aspect herein, the present disclosure is directed to the method of any of aspects 22-38, wherein the medium composition further comprising sodium pyruvate, sodium dodecyl sulfate and/or an antibiotic.

In a 39^{th} aspect, either alone or in combination with any other aspect herein, the present disclosure is directed to the method of the 38^{th} aspect, wherein the antibiotic is cefsulodin.

In a 40^{th} aspect, either alone or in combination with any other aspect herein, the present disclosure is directed to the method of the 38^{th} aspect, wherein the antibiotic is tetracycline.

In a 41^{st} aspect, either alone or in combination with any other aspect herein, the present disclosure is directed to the method of aspects 38, 39, or 40, wherein the antibiotic is present at a concentration of from about 0.0001 g/mL to about 0.001 g/mL

In a 42^{nd} aspect, either alone or in combination with any other aspect herein, the present disclosure is directed to the method of the 38^{th} aspect, wherein SDS is present at a concentration of from about 0.005 to about 0.02 g/mL.

In a 43^{rd} aspect, either alone or in combination with any other aspect herein, the present disclosure is directed to the method of the 38^{th} aspect, wherein sodium pyruvate is present at a concentration of from about 0.005 to about 0.02 g/mL.

In a 44^{th} aspect, either alone or in combination with any other aspect herein, the present disclosure is directed to the method of any of aspects 22-43, wherein the growth medium composition comprises small peptides and/or amino acids and/or cellular extracts.

In a 45^{th} aspect, either alone or in combination with any other aspect herein, the present disclosure is directed to the method of the 44^{th} aspect, wherein the growth medium composition comprises yeast extract and/or casamino acids.

### EXAMPLES

The examples herein sets forth embodiments for inexpensive and expedient systems and methods to detect E. coli in water samples obtained in the field. These examples set forth an approach that can offer detection to reduce the health burdens associated with microbial contamination without the use of expensive or specialized equipment. The exemplary systems and methods described herein allow for quick identification and resolution of water quality risks with the use of common laboratory equipment.

The examples herein may be used to detect E. coli in water samples collected from any variety of sources, such as a household tap, a rain barrel, a recreational water (e.g. beach, swimming pool) cistern, a groundwater source (e.g. dug well, drilled well, bore hole, etc.), surface water (e.g. river, lake, pond, wetland, dam, sea, estuary), or other source of drinking, bathing, or recreational water. The examples set forth utilize the steps of first preparing a liquid media by adding dry goods in an aseptic fashion to a sterile container. The amount of each dry good to be added can be dependent upon the number of water sample tests to be performed. For example, Table 4 identifies the amount of each dry good in grams (g) to be added if performing 80 water sample tests.

TABLE 4

| | g |
|---|---|
| Yeast Extract | 5.0000 |
| Casamino Acids | 2.5000 |
| Sodium Sulfate | 1.2500 |
| Sodium Chloride | 1.2500 |
| Sodium Phosphate Dibasic | 7.5000 |
| Sodium Pyruvate | 0.2500 |
| Potassium Phosphate Monobasic | 3.7500 |
| Ammonium Chloride | 2.5000 |
| Ammonium Sulfate | 2.5000 |
| Magnesium Sulfate | 0.6250 |
| Calcium Chloride | 0.1250 |
| Sodium Dodecyl Sulfate | 0.2500 |
| MUG | 0.1250 |
| Cefsulodin | 0.0150 |
| *Tetracycline | 0.0160 |

In preparing the liquid media, sterile distilled water is mixed with the dry goods to form the liquid media. Accordingly, the sterile container in which the dry goods are added should be of sufficient volume to hold and mix the dry goods and sterile distilled water. In this example, 2.5 mL of distilled water is added for each water sample test to be performed. For example, if 80 water sample tests are to be performed, a sterile container with sufficient volume to hold and mix the dry goods in the amounts shown in Table 1 and 200 mL of sterile distilled water is required. By way of further example, if 100 water sample tests are to be performed, a sterile 500 mL Erlenmeyer flask may be used for holding and mixing the appropriate amount of dry goods and 250 mL of sterile distilled water.

Mixing of the liquid media occurs by placing a sterile magnetic stirring rod into the sterile container and mixing for one-hour on a medium to medium-high setting. During this time, the sterile container is covered or closed to avoid contamination from ambient air. Importantly, excessive foam production during the mixing should be avoided. The presence of excessive foam production indicates that the setting for mixing is too high or vigorous and should be adjusted down. Following mixing, 2.5 mL of the liquid media is added to sterile individual 50 mL centrifuge tubes with the use of sterile micropipette tips. Alternatively, 2.5 mL of the liquid media may be added to other types of sterile sampling containers of 50 mL in volume or larger. Tubes or sampling containers containing the liquid media are then refrigerated until use.

After the media is prepared, it is used to test a water sample for E. coli. 22.5 mL of the water sample is poured or placed into the tube of liquid media, resulting in a 25 mL combined solution of the liquid media and the water sample, and the solution is adequately mixed by capping or covering the tube and inverting the tube a minimum of 30 times. Importantly, vigorous shaking of the tube is avoided as such shaking results in excessive foam or bubbles. After adequate mixing, the solution is poured into a multichannel pipette reservoir. (See FIG. 1).

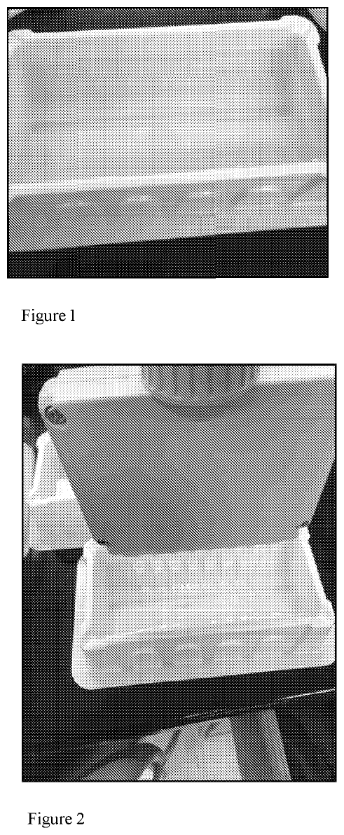

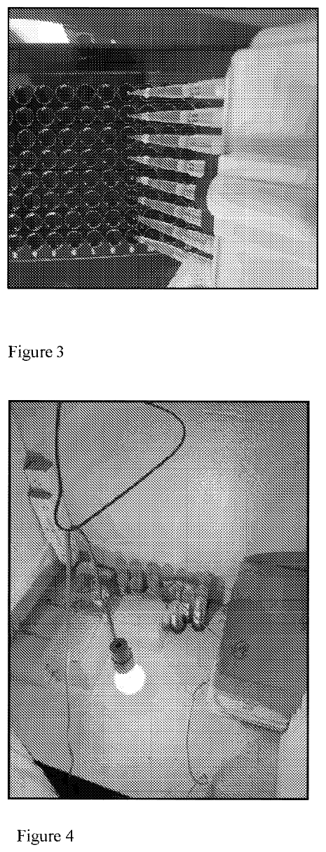

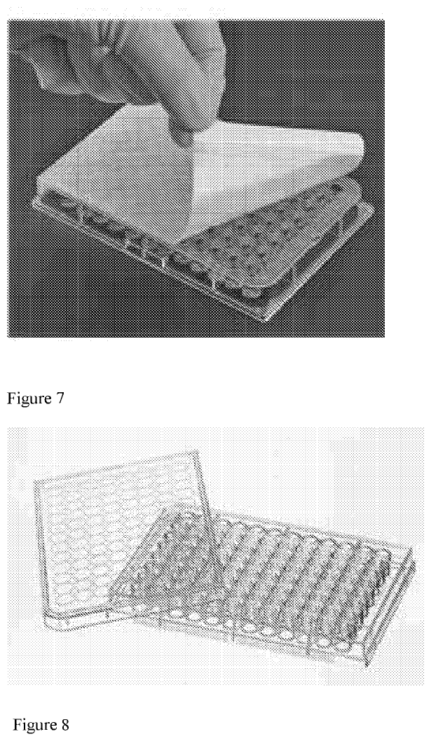

Next, equal amounts of the solution are withdrawn from the reservoir and deposited into individual wells with a multichannel pipette (see FIG. 2) and placed in individual wells. (see FIG. 3). The multiple channel pipette withdraws 200 microliters of the solution per pipette tip and places 200 microliters of the solution into each well of a 96-well plate (in total, 19.2 mL of the solution will be added to the 96-well plate and approximately 5.8 mL will remain in the reservoir). Thereafter, the 96-well plate is sealed or covered by using common laboratory plastic sealing adhesive covers (see FIG. 7) or well lids, e.g., a 96-well plate cover, (see FIG. 8). Unlike other water testing methods, the present disclosure does not require use of expensive sealers or other specialized equipment for sealing. Likewise, by using the multiple channel pipette and pipette tips to evenly place consistent volumes of the solution into each well of the 96-well plate, the present disclosure significantly reduces the amount of the time required for the testing and significantly reduces the likelihood of spillage and cross-contamination as compared to other water testing methods.

After sealing or covering, the individual wells containing the solution are incubated for 18 hours at 35 degrees Celsius. In the absence of a commercial or laboratory incubator, alternative means for incubation may be used, including placing the 96-well plate in any environment capable of maintaining a temperature of 35 degrees Celsius for 18 hours. One example of an alternative means for incubation under the present disclosure includes the use of a 100-watt lightbulb, a thermometer, and an enclosed space wherein the 96-well plate is placed in the enclosed space, the 100-watt lightbulb is lowered from the ceiling and secured at an appropriate height relative to and above the 96-well plate, and the thermometer monitors the temperature at 35 degrees Celsius. (See FIG. 4). In alternative embodiments, the thermometer further comprises an alarm. In alternative embodiments, the 96-well plate, 100-watt lightbulb, and thermometer are placed into an unenclosed area.

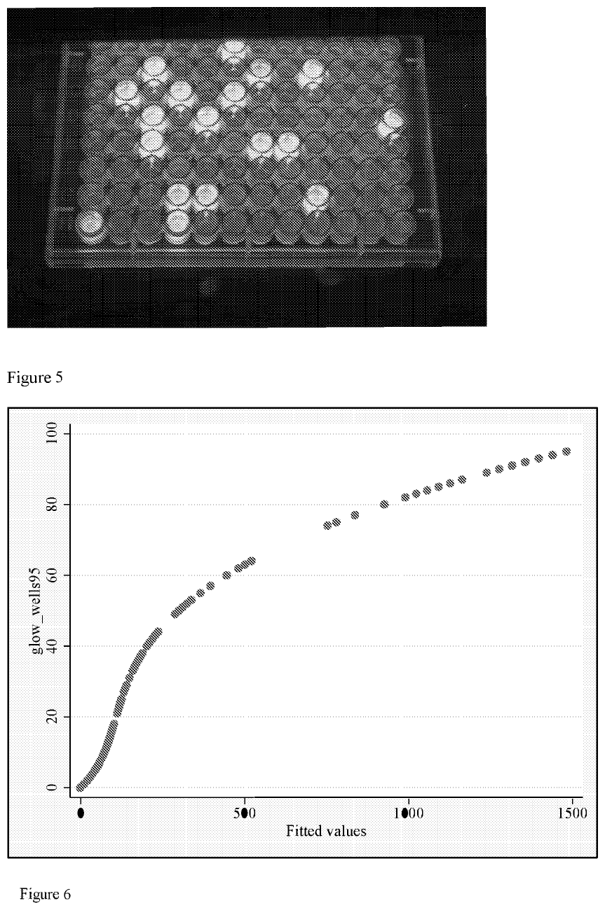

After incubation, the 96-well plate is placed in a dark or dim environment, such as a dark room or cardboard box, wherein a longwave ultraviolet lamp, emitting light at 365 nanometers (nm) is used to shine longwave light over the 96-well plate. Individual wells glowing blue (or turquoise) under the longwave light are counted as presumptively positive E. coli positive wells. (See FIG. 5). Alternatively, a battery operated, handheld black-light may be used as the source of the longwave light.

The number of individual wells glowing blue (or turquoise) under the longwave light is used to determine the E. coli density of the water sample in MPN per 100 mL. The MPN of E. coli per 100 mL of the water sample is determined by utilizing the E. coli density table in Table 5, wherein the identified number of glowing wells, i.e., the number of presumptively E. coli positive wells, corresponds to an MPN per 100 mL.

TABLE 5

| Glowing | MPN per |
|---|---|
| Wells | 100 mL |
| 0 | <5 |
| 1 | 10 |
| 2 | 20 |
| 3 | 29 |
| 4 | 37 |
| 5 | 44 |
| 6 | 51 |
| 7 | 57 |
| 8 | 62 |
| 8 | 62 |
| 8 | 62 |
| 8 | 62 |
| 9 | 68 |
| 10 | 72 |
| 11 | 77 |
| 12 | 81 |
| 13 | 85 |
| 14 | 88 |
| 15 | 92 |
| 16 | 95 |
| 17 | 98 |
| 18 | 102 |
| 21 | 111 |
| 22 | 114 |
| 23 | 117 |
| 24 | 121 |
| 25 | 124 |
| 27 | 132 |
| 28 | 135 |
| 29 | 140 |
| 31 | 148 |
| 33 | 158 |
| 34 | 164 |
| 35 | 169 |
| 36 | 175 |
| 37 | 181 |
| 38 | 188 |
| 40 | 202 |
| 41 | 210 |
| 42 | 218 |
| 43 | 227 |
| 44 | 236 |
| 49 | 287 |
| 50 | 299 |
| 51 | 311 |
| 52 | 324 |
| 53 | 337 |
| 55 | 365 |
| 56 | 380 |
| 57 | 395 |
| 60 | 445 |
| 62 | 481 |
| 63 | 500 |
| 64 | 520 |
| 74 | 753 |
| 75 | 780 |
| 77 | 836 |
| 80 | 926 |
| 82 | 990 |
| 83 | 1023 |
| 84 | 1057 |
| 85 | 1091 |
| 86 | 1127 |
| 87 | 1163 |
| 89 | 1237 |
| 90 | 1276 |
| 91 | 1315 |
| 92 | 1356 |
| 93 | 1397 |
| 94 | 1439 |
| 95 | 1481 |
| 96 | >1481 |

Upon determining the MPN of E. coli per 100 mL of the water sample, the 96-well plate may be discarded or archived as needed. A bleach solution can be used to destroy and inactivate microbes.

### Example 1

One hundred seventy-six water samples were obtained from Madison County, Kentucky, and tested for E. coli using the steps described above and also using Colilert, a commercially available water testing kit sold by IDEXX. Results from the testing methods were compared using the Fractional Polynomial method in Stata 15. (See FIG. 5). The percent variability in the predicted MPN values was 88%, which is a strong value and on par or better than R-Squared values for other commercial water testing kits.

### Example 2

Forty water samples from Elgeyo-Marakwet County in Kenya were obtained and tested for E. coli using the steps described above and also using Colilert, a commercially available water testing kit sold by IDEXX. Results from the testing methods were compared using the Fractional Polynomial method in Stata 15. (See FIG. 5). The percent variability in the predicted MPN values was 94% in Kenya, which is a strong value and on par or better than R-squared values for other commercial water testing kits.

### Example 3

Presumably, tetracycline resistant E. coli was observed by the inventor in water samples obtained from Elgeyo-Marakwet County in Kenya. The steps outlined above were utilized to test water samples under conditions wherein the liquid media was comprised of dry goods and tetracycline and under conditions wherein the liquid media did not comprise tetracycline. There was significantly less presumptively positive E. coli positive wells than in testing involving liquid media that did not comprise tetracycline observed in this arrangement.

The foregoing description of several embodiments has been presented for purposes of illustration. It is not intended to be exhaustive or to limit the application to the precise forms disclosed, and obviously many modifications and variations are possible in light of the above teaching. It is understood that the disclosure may be practiced in ways other than as specifically set forth herein without departing from the scope of the disclosure. Any patents or publications mentioned in this specification are incorporated herein by reference to the same extent as if each individual publication is specifically and individually indicated to be incorporated by reference.

1. A system for determining the presence and/or quantity of contaminants in an aqueous sample comprising a dilution container for diluting an aliquot of the aqueous sample in a medium composition solution comprising a growth medium and a substrate for a contaminant to form a mixture and an incubator for incubating the mixture for a period of time and at a temperature favorable for the contaminant to metabolize the substrate, wherein the growth medium comprises sodium sulfate, sodium chloride (or comparable sources of the ions therein), sodium phosphate, potassium phosphate, ammonium chloride, ammonium sulfate, magnesium sulfate and calcium chloride.

2. The system of claim 1, further comprising a means dividing the mixture into 2 or more separated compartments.

3-5. (canceled)

6. The system of claim 1, further comprising a lid.

7. (canceled)

8. The system of claim 1, wherein the substrate is selected from 4-methylumbeliferyl-β-D-glucuronide, resorufin-β-D-glucuronide, fluorescein di-β-D-glucuronide, 5-(Pentafluorobenzoylamino)fluorescein di-β-D-glucuronide 5-bromo-6-chloro-3-indolyl-β-D-glucuronide, 5-bromo-4-chloro-3-indolyl-β-D-glucuronide, 8-hydroxyquinoline glucuronide, 4-nitrophenyl-β-D-glucuronide, phenolphthalein-β-D-glucuronide, phenolphthalein-β-D-glucuronide sodium salt, or an analog thereof.

9-10. (canceled)

11. The system of claim 1, wherein the growth medium comprises from about 0.01 to about 0.1 g/mL of sodium sulfate, from about 0.01 to about 0.1 g/mL of sodium chloride, from about 0.1 to about 0.5 g/mL sodium phosphate, from about 0.1 to about 0.5 g/mL potassium phosphate, from about 0.1 to about 0.5 g/mL of ammonium chloride, from about 0.1 to about 0.5 g/mL of ammonium sulfate, from about 0.01 to about 0.05 g/mL of magnesium sulfate, and from about 0.001 to about 0.01 g/mL of calcium chloride.

12. The system of claim 1, wherein the medium composition further comprising sodium pyruvate, sodium dodecyl sulfate (SDS) and/or an antibiotic.

13. The system of claim 12, wherein the antibiotic is selected from cefsulodin, ceftazidime, cefttibuten (cedax), cefixime, cefoxitin, cefoxitin sodium salt, cefotaxime, cefotaxime sodium salt, cefoperazone, azlocillin, pipcracillin, tobramycin, and latamoxef (moxalactam), gentamicin, tetracycline, glycylcycline, tigecycline, chlortetracycline, oxytetracycline, demeclocycline, lymecycline, methacycline, minocycline, rolitetracycline, doxycycline, imipenem, doripenem, meropenem, etrapenem, clisatatin or a combination thereof.

14. (canceled)

15. The system of claim 12, wherein the antibiotic is present at a concentration of from about 0.0001 g/mL to about 0.001 g/mL.

16. The system of claim 12, wherein SDS is present at a concentration of from about 0.005 to about 0.02 g/mL and wherein sodium pyruvate is present at a concentration of from about 0.005 to about 0.02 g/mL.

17-18. (canceled)

19. The system of claim 1, wherein the growth medium composition comprises yeast extract and/or casamino acids.

20. (canceled)

21. A method for determining the presence and/or quantity of contaminants in an aqueous sample comprising forming a mixture by diluting a aliquot of an aqueous sample in a medium composition solution comprising a growth medium and a substrate and incubating the mixture for a period of time and at a temperature favorable for the contaminant to metabolize the substrate, wherein the growth medium comprises sodium sulfate, sodium chloride (or comparable sources of the ions therein), sodium phosphate, potassium phosphate, ammonium chloride, ammonium sulfate, magnesium sulfate and calcium chloride and wherein the mixture is dispensed into 96 wells within a single plate.

22. The method of claim 21, further comprising detecting for a metabolite of the substrate by exposing the mixture to ultraviolet light.

23-26. (canceled)

27. The method of claim 21, further comprising counting the wells wherein the metabolite is detected.

28. (canceled)

29. The method of claim 21 any of claims 22-29, wherein the mixture is incubated at a temperature of from about 20 to about 40° C.

30. (canceled)

31. The method of claim 22 21 any of claims 22-31, wherein the mixture is incubated for a period of from about 10 to about 48 hours.

32. (canceled)

33. The method of claim 22 21 any of claims 22-33, wherein the substrate is selected from 4-methylumbeliferyl-β-D-glucuronide, resorufin-β-D-glucuronide, fluorescein di-β-D-glucuronide, 5-(Pentafluorobenzoylamino) fluorescein di-β-D-glucuronide or an analog thereof.

34-35. (canceled)

36. The method of claim 22 21, wherein the growth medium comprises from about 0.01 to about 0.1 g/mL of sodium sulfate, from about 0.01 to about 0.1 g/mL of sodium chloride, from about 0.1 to about 0.5 g/mL sodium phosphate, from about 0.1 to about 0.5 g/mL potassium phosphate, from about 0.1 to about 0.5 g/mL of ammonium chloride, from about 0.1 to about 0.5 g/mL of ammonium sulfate, from about 0.01 to about 0.05 g/mL of magnesium sulfate, and from about 0.001 to about 0.01 g/mL of calcium chloride.

37. The method of claim 22 21 any of claims 22-38, wherein the medium composition further comprising sodium pyruvate, sodium dodecyl sulfate, yeast extract, casamino acids and/or an antibiotic.

38. The method of claim 38 37, wherein the antibiotic is cefsulodin, ceftazidime, cefttibuten (cedax), cefixime, cefoxitin, cefoxitin sodium salt, cefotaxime, cefotaxime sodium salt, cefoperazone, azlocillin, pipcracillin, tobramycin, and latamoxef (moxalactam), gentamicin, tetracycline, glycylcycline, tigecycline, chlortetracycline, oxytetracycline, demeclocycline, lymecycline, methacycline, minocycline, rolitetracycline, doxycycline, imipenem, doripenem, meropenem, etrapenem, clisatatin or a combination thereof.

39. (canceled)

40. The method of claim 38 37 claim 38, 39, or 40, wherein the antibiotic is present at a concentration of from about 0.0001 g/mL to about 0.001 g/mL, SDS is present at a concentration of from about 0.005 to about 0.02 g/mL and sodium pyruvate is present at a concentration of from about 0.005 to about 0.02 g/mL.

41-45. (canceled)
